(19) United States
(12) Patent Application Publication
(10) Pub. No.: US 2022/0044814 A1
(43) Pub. Date: Feb. 10, 2022

(54) DEVICE AND METHOD FOR LOCALISING OR IDENTIFYING MALIGNANCIES

(71) Applicant: ESPIRE TECHNOLOGIES GMBH, Berlin (DE)

(72) Inventors: Konstantin CHERNOV, Berlin (DE); Eugene LEVONYCK, Berlin (DE); Kerstin SCHNURR, Ahrensfelde (DE); Katja WATERSTRADT, Berlin (DE)

(73) Assignee: ESPIRE TECHNOLOGIES GMBH, Berlin (DE)

(21) Appl. No.: 17/507,835

(22) Filed: Oct. 22, 2021

Related U.S. Application Data
(63) Continuation-in-part of application No. PCT/EP2019/060378, filed on Apr. 23, 2019.

(51) Int. Cl. G16H 50/20; G01N 24/10; G06T 7/00; G16B 40/10; G16B 40/20

(52) U.S. Cl. CPC G16H 50/20; G01N 24/10; G06T 7/0012; G16B 40/10; G06T 2207/30068; G06T 2207/10081; G06T 2207/10088; G06T 2207/10116; G16B 40/20

(57) ABSTRACT

Provided herein are methods for identifying and treating a malignancy in a patient. Aspects of the described methods are performed through use of a computing device. The method comprises receiving at the computing device a plurality of spectra acquired from a corresponding plurality of aliquots containing a biophysiological carrier protein. At least one of a concentration of a spin probe and a concentration of a polar reagent varies between the aliquots. The computing device then determines biophysical parameters based on the received spectra and applies at least parts of the received spectra and the biophysical parameters as an input to a trained logistic regression model. The logistic regression model trained to determine a probability of applied input parameters relating to one or more of a plurality of predetermined diseases and/or disease localisations. The trained model is used to determine a probability of the input parameters relating to one or more of said predetermined diseases and/or disease localisations and outputs a result of the determination, which can be used to determine and then provide proper therapeutic treatments.

### CROSS-REFERENCE TO RELATED APPLICATIONS

This is a Continuation-in-Part of International Patent Application No. PCT/EP2019/060378, filed on Apr. 23, 2019, the contents of which are incorporated by reference herein in their entirety.

### FIELD

Embodiments generally relate to methods and devices analysing extracellular fluids that contain a carrier protein, for example serum albumin and, more particularly, may relate to methods and associated devices for analysing the carrier protein to detect indicators of a malignancy and its localisation within the human body. In embodiments such analysis is carried out using electron spin resonance spectroscopy (ESR).

### BACKGROUND

Growth processes of tumours can be accompanied by the secretion of metabolites into the bloodstream. It is known to analyse blood samples to detect the presence of such metabolites. However, many known techniques that seek to detect such metabolites have poor sensitivity and have a strict focus on a limited set of metabolites. Most often known techniques attempt to recognise malignancy of one localization. This can be to the extent that other symptoms of a malignant growth can be detected simultaneously. As a consequence some such other techniques are limited in their suitability as a tool for screening blood samples for the presence of indicators of malignant growth.

### SUMMARY

Described herein are methods for identifying and treating a malignancy in a patient. According to an embodiment, aspects of the described methods are performed in a computing device. The method comprises receiving at the computing device a plurality of spectra acquired from a corresponding plurality of aliquots containing a biophysiological carrier protein, wherein at least one of a concentration of a spin probe and a concentration of a polar reagent varies between the aliquots, determining by the computing device biophysical parameters based on the received spectra and applying by the computing device at least parts of the received spectra and the biophysical parameters as an input to a trained logistic regression model. The logistic regression model is trained to determine a probability of applied input parameters relating to one or more of a plurality of predetermined diseases and/or disease localisations. The computing device using the trained model to determine a probability of the input parameters relating to one or more of said predetermined diseases and/or disease localisations. A result of the determination of a probability is output, which is then used to identify and then treat the identified disease.

### BRIEF DESCRIPTION OF THE DRAWINGS

The present embodiments will be understood and appreciated more fully from the following detailed description taken in conjunction with drawings in which:

FIG. 1 is a schematic diagram of the structure of human serum albumin for use in an embodiment;

FIG. 2 is a schematic diagram of an exemplary procedure for the evaluation of albumin by ESR spectroscopy in accordance with an embodiment;

FIG. 3 is a graphical representation of 16-doxyl stearic acid for use in an embodiment;

FIG. 4 is a schematic diagram of the effect of ethanol concentration on albumin conformation in accordance with an embodiment;

FIG. 5 illustrates a 9.45 GHz ESR spectrum of human serum albumin;

FIG. 6 shows five sub-spectra of the spectrum shown in FIG. 5;

FIG. 7 shows an example of an acquired ESR spectrum;

FIG. 8 shows depicts a system of an embodiment comprising an ESR spectrometer and a computing device; and

FIG. 9 shows a flowchart of a method according to an embodiment;

FIG. 10 is a flowchart of a method of training a logistic regression model.

### DETAILED DESCRIPTION

According to an embodiment there will be provided a method performed in a computing device. The method comprises receiving at the computing device a plurality of spectra acquired from a corresponding plurality of aliquots containing a biophysiological carrier protein, wherein at least one of a concentration of a spin probe and a concentration of a polar reagent varies between the aliquots, determining by the computing device biophysical parameters based on the received spectra and applying by the computing device at least parts of the received spectra and the biophysical parameters as an input to a trained logistic regression model. The logistic regression model is trained to determine a probability of applied input parameters relating to one or more of a plurality of predetermined diseases and/or disease localisations. The computing device using the trained model to determine a probability of the input parameters relating to one or more of said predetermined diseases and/or disease localisations. A result of the determination of a probability is output.

In an embodiment determining the biophysical parameters comprises determining one or more or all of a spectral component from spin probe bound to albumin with a high binding affinity, a spectral component from spin probe bound to albumin with a low binding affinity, a spectral component from free spin probe molecules, a spectral components from free spin probe in micelles and a spectral component from spin probe on lipid-fraction of serum.

In an embodiment the biophysiologial parameters are selected from one or more or all of polarity surrounding a spin label in one or more high affinity spectral components, spin probe ordering, spin probe effective correlation time, spectral intensity and a spectral geometry factor.

The method may further comprise receiving in an ESR spectrometer, a plurality of aliquots containing a biophysiological carrier protein, wherein at least one of a concentration of a spin probe and a concentration of a polar reagent varies between the aliquots, acquiring, for each aliquot, an ESR spectrum and transmitting acquired ESR spectra to said computing device.

The method may further comprise preparing a plurality of aliquots containing a biophysiological carrier protein, wherein at least one of a concentration of a spin probe and a concentration of a polar reagent varies between the aliquots.

According to a further embodiment there is provided a non-transitory storage medium storing program instructions for execution by a processor, the program instructions configured to, when executed by the processor, cause the processor to perform a method as described herein.

According to a further embodiment there is provided an analysis system comprising a processor, memory storing program instructions suitable for execution by said processor and a trained logistic regression model, the logistic regression model trained to determine a probability of applied input parameters relating to one or more of a plurality of predetermined diseases and/or disease localisations, an input interface for receiving spectral data and an output interface for outputting a computation result. The program instructions are configured to cause the processor to, when executed by the processor, receive a plurality of spectra acquired from aliquots containing a biophysiological carrier protein, wherein at least one of a concentration of a spin probe and a concentration of a polar reagent varies between the aliquots, determine biophysical parameters based on the received spectra, applying by the computing device at least parts of the received spectra and the biophysical parameters as an input to the trained logistic regression model, use the trained model to determine a probability of the input parameters relating to one or more of said predetermined diseases and/or disease localisations; output a result of said determination of a probability using said output interface.

In an embodiment the program instructions, when executed by the processor, cause the processor to determine, as part of the determining of biophysical parameters, one or more or all of a spectral component from spin probe bound to albumin with a high binding affinity, a spectral component from spin probe bound to albumin with a low binding affinity, a spectral component from free spin probe molecules, a spectral components from free spin probe in micelles and a spectral component from spin probe on lipid-fraction of serum.

In an embodiment the biophysiologial parameters are selected from one or more or all of: a polarity surrounding a spin label in one or more high affinity spectral components, spin probe ordering, spin probe effective correlation time, spectral intensity and a spectral geometry factor.

The system may further comprise an ESR spectrometer, the ESR spectrometer configured to receive samples for spectral analysis and comprising an output interface, the output interface of the ESR spectrometer communicatively connectable or connected to the said input interface for receiving spectral data.

In another embodiment there is provided a method of training a logistic regression model for determining a probability of applied input parameters relating to one or more of a plurality of predetermined diseases and/or disease localizations. The method comprises
a) providing in a computing device an untrained logistic regression model comprising model parameters;
b) using the model to predict a disease type and/or localization for a training data set;
c) updating said model parameters based on a prediction error and a known disease type and/or localization for said training data set;
d) repeating steps (b) and (c) using further training data sets.

Reference will now be made in detail to the exemplary embodiments implemented according to the present disclosure, the examples of which are illustrated in the accompanying drawings. Wherever possible, the same reference numbers will be used throughout the drawings to refer to the same or like parts.

In patients with cancer, proteins released by tumour cells and their breakdown products can bind to serum albumin in the transport system of blood. Albumin is the main component in the transport system of blood, enabling the transport of fatty acids, tryptophane, bilirubin, calcium, steroid hormones, and other functionally significant active substances to the target cell, and being involved in binding and distribution of a variety of toxins (including those of endogenous origin). Albumin is a polypeptide (molecular weight 68,000) comprised of 585 amino acids and having a high binding capacity for exogenous (drugs) and endogenous substances. Fatty acids are the primary physiological ligand of albumin.

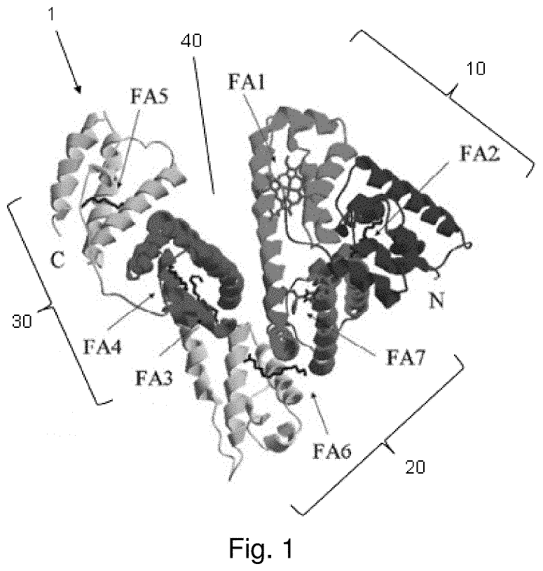

FIG. 1 is a schematic diagram of the structure of human serum albumin (HSA) 1 for use in an embodiment. The structure of HSA 1 comprises three homologous domains 10, 20, 30 that fold into a heart-shaped albumin molecule 1. X-ray crystallographic analysis of HSA has identified at least seven distinct fatty acid binding sites (FA1-FA7) that are located in various parts of the protein (Bhattacharya et al. J Mol Biol. 2000 Nov. 10; 303(5):721-32) and which show relative affinities for fatty acids (Simard et al. J Mol Biol. 2006 Aug. 11; 361(2):336-51). The FA binding sites have certain common features: in each case, the hydrocarbon chain of the fatty acid is accommodated in a long and narrow hydrophobic well, while the carboxyl moiety is located near basic or polar residues (Curry et al. Nat Struct Biol. 1998 September; 5(9):827-35). These FA binding sites (FA1-FA7) represent specific (primary) hydrophobic binding sites for fatty acids in HSA. In addition, non-specific (secondary) hydrophobic binding sites for fatty acids on serum albumin have been identified. These non-specific hydrophobic binding sites are believed to be located in the hydrophobic area 40 between the albumin domains 10, 20, 30 (Gurachevsky et al. Biochem Biophys Res Commun. 2007 Sep. 7; 360(4):852-6). It is believed that there is migration of fatty acids between the non-specific binding sites in the hydrophobic area 40 of HSA and the specific FA binding sites (FA1-FA7) and vice versa and that this process is important for the transport properties of albumin.

As mentioned above, tumour-derived proteins and peptides can bind to serum albumin. It is believed that albumin plays an important role in modulating the serum concentrations of these products by sequestering and protecting them from catabolism, which significantly amplifies their concentration in circulation. The binding of these products to albumin can result in changes in the transport properties of albumin, which can be determined using non-covalent spin-labelling of albumin combined with electron spin resonance (ESR) spectroscopy, also known as electron paramagnetic resonance (EPR) spectroscopy. ESR spectroscopy is therefore a useful tool for evaluating the structural and functional changes that can occur to albumin following the binding of various ligands, including tumour-derived proteins and peptides.

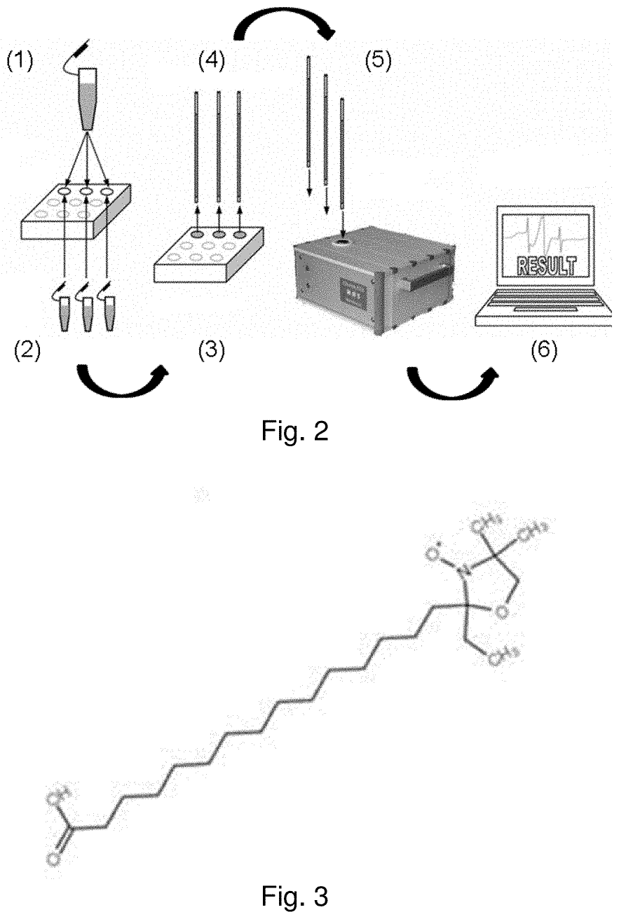

FIG. 2 is a schematic diagram of an exemplary procedure for the evaluation of albumin by ESR spectroscopy in accordance with an embodiment. The procedure is also described in international patent application WO 01/65270, the entirety of which is incorporated herein by reference and comprises the following steps.

In step (1), a sample aliquot containing albumin is placed into a container. In one embodiment, the sample is a serum sample. In one embodiment, the sample is a blood sample or a drug or product that contains albumin. For example, in one embodiment, the sample is a commercial solution comprising a human or bovine albumin preparation. In one embodiment, three sample aliquots are used. However, in an alternative embodiment, fewer or more sample aliquots are used; for example, 1, 2, 4, 5, 6, 7 or 8.

In one embodiment, prior to step (1), a pre-analytical phase is conducted in order to preserve the original (native) conformational state of albumin as it is comprised within the sample to be evaluated. It is preferred that one or more of the following considerations are taken into account.

It is preferred that serum and EDTA-plasma samples are used. It is preferred that preparations with preservatives or anticoagulants (such as heparin), which can bind albumin and modify its native conformational state, are avoided. In embodiments where the sample is whole blood, it is preferred that the process of haemolysis is avoided. In one embodiment, to reduce haemolysis, centrifugation of whole blood for serum sampling is done within one hour of sampling at room temperature. In one embodiment, centrifugation is performed for 10 minutes at 1000 to 1500 g. In one embodiment, vacuum sampling systems are avoided during sampling of whole blood as it is believed that this can influence the stability of certain whole blood and serum samples over time. It is preferred that freezing of the whole blood sample before serum sampling is avoided as it is believed that this disturbs the native conformational state of the components of whole blood. In one embodiment, whole blood is stored or transported cooled for a maximum of 24 hours before centrifugation. In one embodiment, separated serum or EDTA-plasma is stored before analysis in a frozen state at a temperature not higher than −28° C. (due to on-going biochemical prepossess that are believed to occur even in frozen serum). It is preferred that samples are unfrozen only once and that this is shortly before use in the procedure. In one embodiment, the maximum time between defrosting the sample and measurement in the ESR spectrometer is 40 minutes. In embodiments where the sample is an albumin-containing preparation such as a commercial albumin solution or a control sample, the recommendations of the manufacturer regarding the preparation of a control sample, an in particular the dilution of lyophilised albumin, are considered. It is preferred to have enough material of each sample for at least one controlling repetition of the measurement.

It is preferred that icteric and lipemic samples are eliminated from analysis, as the direct evaluation of the conformational state of albumin in such samples is complicated. In such cases repetition of the analysis at a later stage may be required to obtain precise results. Furthermore, it is preferred that samples from donors that fall within certain categories are excluded. In one embodiment, these categories comprise patient suffering from an acute inflammation process, patients less than 21 days post-surgery of a defined invasive procedure, as defined in the NCI dictionary (https ://www.cancer.gov/publications/dictionaries/cancer-terms/def/invasive-procedure) and/or the donor has taken a precluded drug in the past 14 days. In one embodiment, a precluded drug is a drug listed in Table 1. It is believed that these drugs influence the conformational state of albumin at a therapeutic dose.

TABLE 1
Excluded substances

cortisone
octanoates
salicylates
warfarine
lysophospatic acid
lysophosphatidylcholine
tretinoine
valproate
Letrozol ®
Diclofenac ®
Tamoxifen ®

In step (2) of the exemplary procedure of FIG. 2, each sample aliquot is mixed with a spin probe in a polar reagent. A spin probe (also known as a spin label) is an organic molecule that possesses an unpaired electron and has the ability to bind to another molecule. In the present embodiment, the spin probe is 16-doxyl stearic acid (a graphical representation is shown in FIG. 3). In an alternative embodiment, the spin probe is an alternative spin-labelled fatty acid, preferably a doxyl stearic acid, and is one of 5-, 7-, 12- or 16-doxyl stearic acid or 16-doxyl stearate (Soduim). In one embodiment, it is a doxyl lauric acid, preferably 7-doxyl lauric acid. In one embodiment, any spin-labelled compound which can undergo specific binding to albumin (including a further spin-labelled fatty acid, a steroid hormone or a heterocyclic hydrocarbon) is used as a spin probe. Hydrophobic compounds labelled with nitroxyl radicals are used in one embodiment. In the present embodiment, the polar reagent is ethanol. In an alternative embodiment, an alternative alcohol or DMSO is used. It is preferred to use a C₁-C₆ alcohol. In the present embodiment, the polar reagent acts as a solvent for the spin probe and acts to modify the polarity of the mixture. As will be described in further detail below, in the present embodiment, the three sample aliquots vary in the concentration of albumin and spin probe. In addition, the strength of hydrophobic interactions in the albumin-spin probe mixture is varied through use of differing amounts of polar reagent. In alternative embodiments, fewer or more than three different concentrations of albumin, spin probe and/or polar reagent are used; for example, 1, 2, 4, 5, 6, 7 or 8. Varying the concentration of spin label that is added to albumin and changing the ionic strength of the spin label mixture enables ESR spectra to be generated under different conditions. Suitable spin probes and polar reagents are known and some suitable spin probes and polar reagents are disclosed in WO 01/65270, the entirety of which is incorporated herein by this reference.

In the present embodiment, three different concentrations of spin probe of 3.5, 5.8 and 7.5 mmol/l are mixed with 50 μl of each serum sample aliquot in the volumes of 10, 12 and 14 μl respectively. In one embodiment, the mean value of the ratio of spin probe concentration to albumin concentration is 2.5±0.5 and, starting from this mean value, at least two additional concentrations are selected whose deviation from this mean value is no less than 1.0. The concentrations of polar reagent to be added are selected in such a way that the mean value of the final concentration of polar reagent in the aliquots is (0.6±0.25)×Cp, wherein Cp represents the critical concentration of polar reagent, surpassing of which would result in denaturing of the albumin, and, starting from this mean value, at least two additional concentrations of polar reagent are selected, whose deviation from this mean value is at least 15%. Further details on the proportions of spin probe, albumin and polar reagent are described in US 2003/170912 A1/U.S. Pat. No. 7,166,474, which are incorporated herein by reference in their entirety.

Without wishing to be bound by theory, by varying the concentration of spin probe and the concentration of polar reagent, it is believed that a combination of concentrations can be produced, which enables the transport properties of the albumin to be detected at different stages, namely, the physiological state during binding of hydrophobic compounds such as fatty acids (low concentration of spin probe and low concentration of polar reagent), the physiological state during transport of hydrophobic compounds through the vascular system (high concentration of spin probe and low concentration of polar reagent), and the physiological state during delivery (release) of hydrophobic compounds to the target cells (high concentration of spin probe and high concentration of polar reagent).

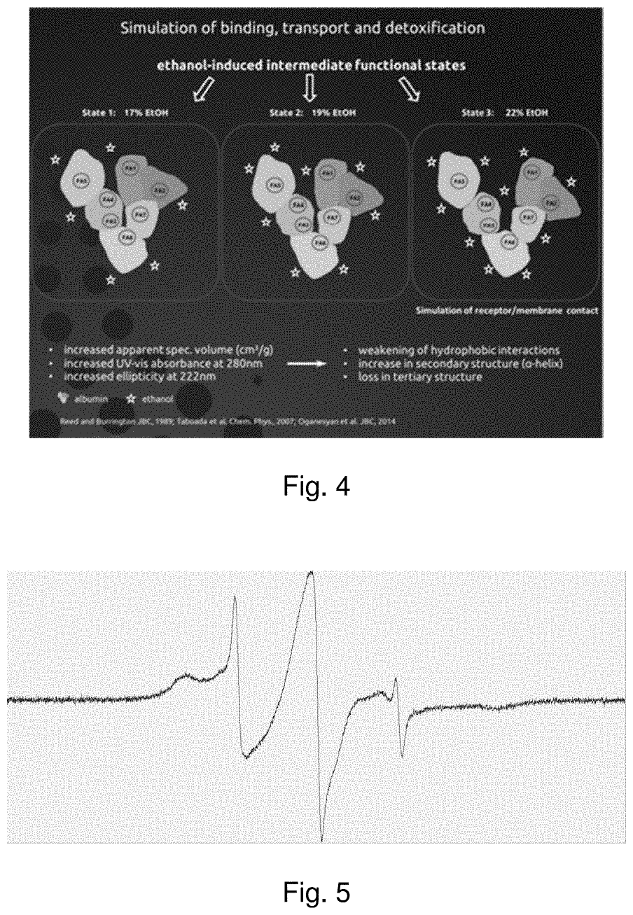

FIG. 4 is a schematic diagram of the effect of ethanol concentration on albumin conformation in accordance with an embodiment. At increasing concentrations of ethanol, it is believed that there is a conformational change in the albumin molecule and a weakening of hydrophobic interactions. This state is believed to result in dissociation of ligands bound to albumin, including the spin probes used in embodiments of the present invention. Therefore, it is believed that by varying the concentration of the polar reagent, specific conformational changes in the albumin molecule can be induced, which enable ESR spectra to be generated under different conditions.

In step (3) of the exemplary procedure of FIG. 2, the mixture of the sample, spin probe and polar reagent is incubated. In the present embodiment, the incubation period is 10 minutes at 37° C. and at the physiological pH of blood. In an alternative embodiment, the incubation period is less or more than 10 minutes; for example, from 7 to 15 minutes. In an alternative embodiment, two or more different temperature values of the samples ranging between 15 and 45° C. and/or two or more different pH values of the serum samples ranging from 7.5 to 3.5 are used.

In step (4) of the exemplary procedure of FIG. 2, the mixture is taken up by capillary tubes. In step (5), an ESR spectrometer is used to measure ESR spectra from each capillary tube. In step (6), the ESR spectra are analysed and results are calculated.

To perform ESR spectroscopy the capillary tube is inserted into an ESR spectrometer. Suitable ESR spectrometers are available from MedInnovation GmbH (Berlin, Germany), for example models EPR 01-08, MS-400 and Espire-5000. ESR spectroscopy is a known technique that does not need to be discussed in detail in the present disclosure. Briefly, however, ESR spectra are acquired by exposing the sample to a strong static magnetic field. The application of the static magnetic field causes the separation of free electrons into two spin states. The application of microwave energy at the correct frequency causes spins to transition between the states. The microwave energy absorbed in this transition is measurable. It is either possible to maintain the microwave frequency constant and change the strength of the static magnetic field while monitoring the amount of microwave energy that is being absorbed or keeping the static magnetic field unchanged while sweeping the microware frequency over a range whilst monitoring energy absorption. The exact static magnetic field strength/microwave frequency combination that provides the correct amount of energy required by a spin to transition between the spin states depends on the chemical environment of the spin. It will be understood that a given spin probe consequently requires different amounts of energy to accomplish this transition, depending on the whether or not the spin probe is bound to albumin and indeed on the manner in which it is bound to albumin. Consequently ESR based methods can distinguish between unbound and bound spin probes. Moreover, ESR is able to distinguish between spin probes located at different binding sites on the albumin complex or between different spin probes in different unbound conditions. As such ESR is a powerful tool for assessing the binding conditions found on a molecule, in the embodiment on serum albumin.

An EPR spectrum can be generated by tracking the amount of microwave energy absorbed as the strength of the static magnetic field is ramped up or down over a predetermined range and by forming the first derivative of the tracked absorption spectrum. As the strength of the static magnetic field changes, so does the separation between the two spin states of the free electron. The separation between these two spin states does not only depend on the applied static magnetic field strength but also on the chemical environment within which the spin is located. By observing the amount of energy absorbed at each given static magnetic field strength conclusions can be drawn regarding the amount of spin probe present in a binding state that produces a separation in the energy.

Other X-Band EPR spectrometers (operating with a microwave frequency of approximately 9-10 GHz, can be also used in embodiments. The sample can be maintained at 37° C. during the measurement process to mimic physiologic conditions.

Particular binding sites of spin probes on albumin produce spectral ESR patterns all signatures that can be detected. Analysis of such signatures allow determination of the amount of spin probe bound to the various binding sites. This said, it is not unusual for ESR patterns are generated by the influence different binding sites have on spin probes to overlap with each other.

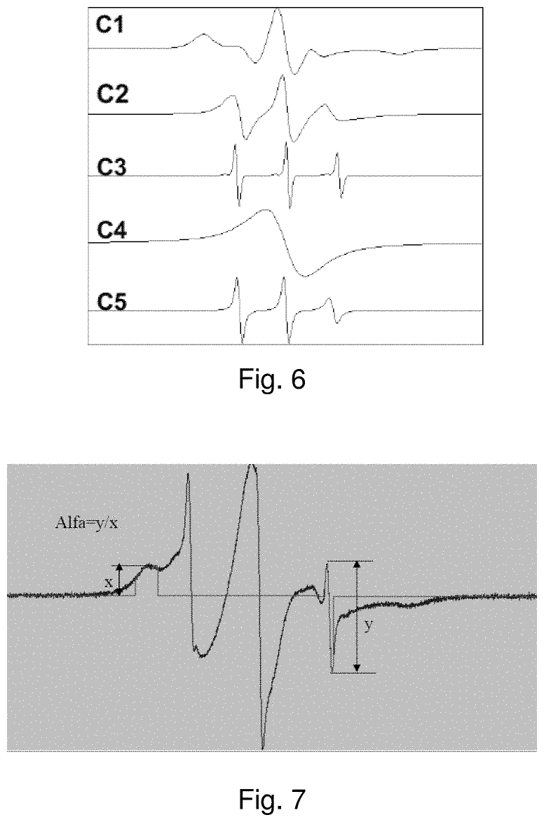

FIG. 5 illustrates a 9.45 GHz ESR spectrum of human serum albumin. This spectrum consists of a number of overlapping sub-spectra. The measured spectrum shown in FIG. 5 can, for example, be decomposed into five sub-spectra, as shown in FIG. 6. The spectral components mentioned in FIG. 6 described in Table 2 and relate to spin probes respectively bound to albumin with high and low affinity or are present in the serum in the states mentioned in the table.

TABLE 2
Spectral components

| | |
|---|---|
| C1 | low motion albumin bound component |
| | with a high affinity |
| C2 | low motion albumin bound component |
| | with a lower affinity |
| C3 | free spin probe (16-doxyl stearic acid) |
| | molecules |
| C4 | free spin probe (16-doxyl stearic acid) |
| | in micelles |
| C5 | spin probe (16-doxyl stearic acid) |
| | bound on lipid-fraction of the serum |

C1 represents the proportion of the fatty acid spin probe bound to the specific binding sites in albumin (i.e. at FA binding sites FA2, FA4 and FA5). C2 represents the proportion of fatty acid spin probe bound to the non-specific binding sites, binding sites 1, 3, 6 and 7 in the hydrophobic area of albumin. C3 to C5 represent unbound spin probe molecules (i.e. spin probes that are not bound to albumin). More specifically, C3 represents the proportion of spin probe molecules that are unbound and present free in the sample; C4 represents the proportion of spin probe molecules that are aggregated into clusters of fatty acid micelles; and C5 represents the proportion of spin probe molecules that are associated with or bound to lipoproteins in the serum sample.

The contributions individual spectral components make to the measure spectrum can be determined by simulating the individual spectral components and then fitting the simulated components to the measured spectrum, adjusting the magnitude and phase of the individual spectral components until a fitting criterion is fulfilled. This criterion may, for example, be the minimisation of the root mean square error between the measured spectrum and the sum of the individual simulated spectral components as weighted by the above mentioned phase and magnitude term. The magnitudes and phases the spectral components have after this fitting indicate the concentration of spin probes in the various binding states set out in Table 2. In an embodiment the spectral components are simulated in the manner described by Andrey Gurachevsk, Ekaterina Shimanovitch, Tatjana Gurachevskaya, Vladimir Muraysky (2007) Intra-albumin migration of bound fatty acid probed by spin label ESR. Biochemical and Biophysical Research Communications 360 (2007) 852-856, the entirety of which is incorporated herein by this reference. The details of the method of simulation the spectral components need not be discussed in detail in the present disclosure.

In an embodiment the simulated spectral lines are fitted to the measured ESR spectrum using a least squares fit. Alternatively, maximum likelihood estimation may be used. Based on the fitting of the five simulated components C1 to C5 as defined in Table 2, the relative concentrations of components C1 to C5 for each of the three above described aliquots (hereinafter also referred to by the short-hand A, B, C) are determined. This can be done in the manner described in WO 2000/004387A3, the entirety of which is incorporated herein by reference.

Further biophysical parameters can also be determined based on the measured spectra. These include:
The polarity (hydrophilicity) surrounding the spin label for the first/high affinity spectral component C1 and for the second/low affinity spectral component C2
Spin probe ordering
Spin probe effective correlation time
Intensity
Geometry factor Alpha

The polarity (hydrophilicity) surrounding the spin label for the first/high affinity spectral component C1 and for the second/low affinity spectral component C2 may be referred to as H₁ (for the C1 component) and H₂ (for the C2 component) and are, in some sources, referred to as “P”. P is defined as:

$$P = \frac{A - {A}_{H}}{{A}_{W} - {A}_{H}} A = ( {A}_{∥} + 2 {A}_{⊥} ) \text{/} 3$$

where A_{⊥,∥}—are hyperfine splitting constant respectively perpendicular or parallel to the axis of external magnetic field as applicable C1 and C2 (the high and low affinity binding sites have different hyperfine splitting constants associated with them), A_{H} is the hyperfine splitting constant for a spin probe in a hydrophobic medium, and A_{W} is the hyperfine splitting constant for a spin probe in a hydrophilic medium (see Muraysky, V., Gurachevskaya, T., Berezenko, S., Schnurr, K., Gurachevsky, A. (2009): Fatty acid binding sites of human and bovine albumins: differences observed by spin probe ESR., Spectrochim Acta A Mol Biomol Spectrosc, 74, 42-47).

Spin probe ordering is associated with the angle of the spin label axis precession for the first and for the second component, S_{C1} (for component C1) and S_{C2} (for component C2), corresponding:

$$S = \frac{{A}_{∥} - {A}_{⊥}}{{A}_{Z}^{0} - ( {A}_{X}^{0} + {A}_{Y}^{0} ) \text{/} 2} \cdot \frac{{A}_{Z}^{0} + {A}_{X}^{0} + {A}_{Y}^{0}}{{A}_{∥} + 2 {A}_{⊥}}$$

Spin probe effective correlation time, T₁ (for component C1) and T₂ (for component C2). T can also be referred to as “τ”):

$$τ = 4 .17 \cdot {10}^{- 10} \cdot {( 1 - \frac{{A}_{∥}^{0}}{{A}_{∥}^{0}} )}^{- \frac{3}{2}} \cdot \frac{32}{{A}_{∥}^{0}}$$

The intensity (or Intens)—is the absolute intensity of corresponding ESR-Spectrum A, B, C in relative units of microwaves-extinction, determined as double-integral of the detected ESR-Spectrum.

FIG. 7 shows an ESR spectrum, A, B or C. The geometry factor alpha is calculated as the ratio of the amplitude of the left-most spectral component of the ESR spectrum and the magnitude of the last but one spectral component on the right hand side of the ESR spectrum. These two peaks are related by their g-factors. Alfa is the ratio of intensities at the position of the high-field line of unbound 16-doxil-straeic spin label relative to the spectral intensity at the position of low-field line of the bound 16-DSA in the albumin binding sites with high affinity.

T_{1Freed} is the correlation time for the first motional component calculated as follows:

$$τ = 5.4 * 1 {0}^{- 10} * {( 1 - ( \frac{{A}_{max}}{{A}_{zz}} ) )}^{- 1.36}$$

where A_{zz}=33,6G is the constant of hyperfine splitting projection for the first component onto Z-axis.

T_{2Podolka} is the correlation time for the second motional component calculated as follows:

$${T}_{2 Podolka} = 5.95 * Δ {H}_{0} * ( \sqrt{\frac{{I}_{0}}{{I}_{+ 1}}} + \sqrt{\frac{{I}_{0}}{{I}_{- 1}}} - 2 ) * 1 {0}^{- 10}$$

where I₋₁, I₀ and I₊₁ are corresponding peak-to-peak high-field, middle-field and low-field intensities of the C2 component of the ESR-spectrum.

T_{GlobFreed} is the correlation time for the albumin globule, calculated as T_{1Feed}/(S1)², wherein S1 is the ordering factor for the first motional component C1.

AppDissConst is the apparent constant of dissociation of the spin-label with albumin determined by:

$${K}_{0} = \frac{{k}_{- 1}}{{k}_{1}} = \frac{[ R ] [ L ]}{[ R L ]} = \frac{[ {R}_{0} - b ] f}{b}$$

where R is the concentration of albumin, L is the concentration of the spin probe and RL is the concentration of the complex of both agents. R₀ is the estimated total binding capacity of albumin with the spin probe, determined by multiplying the concentration of albumin measured in the blood sample by 7 (the number of binding sites of fatty acids on albumin), f is the concentration of free (unbound) spin probe (determined (three times for A, B and C respectively) by multiplying C3 with concentration of the spin probe) and b is the concentration of bound spin probe (determined (again, three time for A, B and C respectively) by adding C1 and C2 and multiplying it with the concentration of the spin probe probe).

Correlation time τ₁ of the albumin globule rotation and the correlation time τ₂ of the spin probe motion relative to the globule (with the axis precession within the angle θ) are calculated from the following equations:

$${τ}^{- 1} = {τ}_{1}^{- 1} + {τ}_{2}^{- 1} τ = {( \frac{3 {cos}^{2} θ - 1}{2} )}^{2} {τ}_{1}$$

Transport parameters of albumin (binding efficiency BE, detoxification efficiency DTE, real transport quality RTQ) are calculated from the following equation of binding constant for the three different concentrations of the ethanol:

$${C}_{Unbound\_DSA} = \frac{1}{{K}_{B}^{′}} * \frac{{C}_{e t h}}{{C}_{e t h}^{0} - {C}_{e t h}} * \frac{{C}_{Bound\_DSA}}{{C}_{Binding\_sites} - {C}_{Bound\_DSA}}$$

where Cₑₜₕ is the Ethanol concentration in the current sample, Cₑₜₕ⁰ is the Ethanol concentration of ethanol when 16-DSA completely dissociates from albumin and K′_{B} is the Albumin binding constant for LCFA at Cₑₜₕ=½*Cₑₜₕ⁰.

The above discussed correlation times derivable from the ESR spectra provide information on the mobility of a spin probe attached at the respective binding sites and the affinity of protein for the spin probe. In addition, the dipolar interactions between spin probes bound to different parts of the protein can also be measured. Changes in the mobility and binding affinity of a spin probe, and the distribution of the spin probe on the albumin molecule, allows the functional and structural properties of a protein to be assessed. Comparison of the changes that occur to the mobility, binding affinity, and distribution of a spin probe on albumin in normal healthy individuals with those changes observed in patients with cancer and some other disease states can reveal unique alterations. This information can be of value in the diagnosis and monitoring of diseases, such as cancer.

The ESR spectra acquired from the above discussed three aliquots are used as input parameters for this process, in addition to one or more or all of the above discussed biophysical parameters, as relevant to the aliquots. As discussed above, whilst in embodiments described herein three aliquots are used, a different number of aliquots may instead be used.

The spectra and biophysical parameters can be combined in a row vector as follows:

x=(1, \tilde{S}₁^{A}, . . . , \tilde{S}_{Ñ}_{_{A}}^{A}, \tilde{S}₁^{B}, . . . , \tilde{S}_{Ñ}_{_{B}}^{B}, \tilde{S}₁^{C}, . . . , \tilde{S}_{Ñ}_{_{C}}^{C}, [symbol] ^{A}₁, . . . , [symbol] ^{A}_{\tilde{L}}_{_{A}}, [symbol] ^{B}₁, . . . , [symbol] ^{B}_{\tilde{L}}_{_{A}}, [symbol] ^{C}₁, . . . , [symbol] ^{C}_{\tilde{L}}_{_{A}})

wherein \tilde{S}₁^{A}, . . . , \tilde{S}_{Ñ}_{_{A}}^{A}, \tilde{S}₁^{B}, \tilde{S}_{Ñ}_{_{A}}^{A}, \tilde{S}₁^{B}, . . . , \tilde{S}_{Ñ}_{_{B}}^{B} and \tilde{S}₁^{C}, . . . , \tilde{S}_{Ñ}_{_{C}}^{C} are the complete ESR spectra acquired for the three aliquots (as indicated by superscript A, B and C respectively) and wherein [symbol] ^{A}₁, . . . , [symbol] ^{A}_{\tilde{L}}_{_{A}}, [symbol] ^{B}₁, . . . , [symbol] ^{B}_{\tilde{L}}_{_{A}} and [symbol] ^{C}₁, . . . , [symbol] ^{C}_{\tilde{L}}_{_{A}} are the above described biophysical parameters as applied to the three aliquots (again as indicated by superscript A, B and C respectively). It will be appreciated that, whilst the parameters are individual values the spectra are a series of values that, jointly, represent individual spectra. Such spectral data points may, for example, be determined by normalizing the acquired ESR spectra by their intensity and g-factor positioning. Normalisation by intensity is, in an embodiment, done by dividing of every point of each experimental spectrum by the value of the spectrum's own double integral. Normalisation by g-factor can be achieved by placing a predetermined absorption peak (for 16-Doxilstearate the middle absorption peak is chosen in an embodiment) in the center of the spectral frame. Data points extending beyond the spectral frame can be trimmed. A spectral frame may have a predetermined number of data points spaced by a predetermined resolution. If the resolution/spacing achieved by experiment differs from the spacing used by the trained model described below spectral data points with the appropriate spacing can be determined by interpolation.

A spectrum may have several hundred or several thousand data points. These normalized data points form input \tilde{S}₁^{A}, . . . , \tilde{S}_{Ñ}_{_{A}}^{A} for aliquot A, \tilde{S}₁^{B}, . . . , \tilde{S}_{Ñ}_{_{B}}^{B} for aliquot B and \tilde{S}₁^{C}, . . . , \tilde{S}_{Ñ}_{_{C}}^{C} for aliquot C. Should more than three aliquots be used in an embodiment then a correspondingly larger number of spectra will be presented as input for the method. The total length of the vector 1×n.

The learned parameter of the method is the matrix θ∈R^{n×K}., where K is the number of possible output classes. We denote the columns of this matrix by θ⁽ⁱ⁾,i=1̅,̅K̅. This learned parameter has been trained based on clinical data with a known diagnosis and for which input x had been determined.

Logistic regression evaluates the probability that an object with the feature vector x belongs to the class

$$k as \frac{{e}^{x \cdot {θ}^{( k )}}}{{{Σ}^{K}}_{j = 1} {e}^{x \cdot {θ}^{( j )}}} .$$

When classifying a class number is calculated by the formula:

k(x)=arg maxᵢ(τ·θ⁽ⁱ⁾)

From row-vectors s of the attributes corresponding to the spectra of the training set the following matrix: X∈R^{m×n}. xᵢ—is the i row of the matrix X, y∈R^{m×1}—column-vector, yᵢ—the number of the class of an object, which corresponds to the i row of the matrix X.

When training, in accordance with the maximum likelihood method, the matrix θ is calculated, minimizing the function J

$$( θ ) = - C \cdot [ {\sum }_{i = 1}^{m} {\sum }_{k = 1}^{K} 1 \{ {y}_{i} = k \} ln \frac{{{e}^{{x}_{i} \cdot }}^{{θ}^{( t )}}}{{{Σ}^{K}}_{j = 1} {e}^{{x}_{i} \cdot {θ}^{( j )}}} ] + {‖ θ ‖}^{2}$$

C is a regularization parameter.

The above learned parameters were acquired by training the logistic regression method based on spectra acquired in the above discussed manner for a population of 715 patients with known disease types and location within the human body. The patients were group in clusters of colo-rectal cancer, other entero-gastrologic cancers, gynecologic cancers, kidney cancers, leukemia, lung cancer, lymphoma, mamma, pancreas, prostate, stomach, cancers with multiple localization and other cancers. The method of an embodiment was tested on 36 different types of localisations, aggregated in the 13 groups mentioned here. These 13 groups moreover include both solid and hemoblastosis.

Testing the trained method using test spectra (again acquired in the manner discussed above) of patients for which disease type and localisation are also known, it was found, that the method allows reliable identification and localization of malignant processes. The diagnostic sensitivity and specificity for identification and localization of all types of lymphoma as well as of pancreatic cancer of was found to be between 80 and 90% as shown in Table 2:

| Localization | mean | mean |
|---|---|---|
| of malignancy | sensitivity | specificity |
| Lymphoma (incl. Hodkins | 0.87 | 0.93 |
| Lymphoma, non-Hodkins | | |
| Lymphoma, Plasmacytoma, | | |
| T-cell prolymphocytic | | |
| leukemia and Mature B | | |
| cell neoplasm) | | |
| Pancreas | 0.74 | 0.94 |
| Colo-Rectal | 0.59 | 0.90 |
| Leukemia | 0.62 | 0.91 |

It will be appreciated from the above that the localisation method of the embodiment is widely applicable to a large number of localisation tasks.

In a particular example, a study was performed with healthy people and people suffering from different diseases (group 1: disease A (e.g. breast cancer), group 2: disease B (e.g. colon cancer), group 3 . . . ). ESR parameters have been detected and “parameter patterns” for healthy people, disease-A-people, disease-B-people, were determined. These patterns of the different groups overlap partially, it was not possible to determine a pattern such as “disease A: yes; all other diseases: no”. It was rather: “disease A is more likely than disease B (e.g. A: probability 80%, B: probability 60%)”.

For a patient with an unknown disease, the spectra are measured and parameters are determined (parameter pattern) and compared with the “known parameter patterns” associated with the different diseases. Based on this comparison it is possible to determine a probability that the patient suffers from a particular disease. However, as described, all other diseases cannot be completely excluded. Therefore, the present described methods determine probabilities for all diseases (disease A, B, C, . . . , X).

The determination of a particular disease is then a result of a selection process. In a particular embodiment, one might only consider diseases with a probability greater than a particular value (e.g. probability greater than 50%). In another particular embodiment, a selection strategy could be that always the 3 diseases with the highest probabilities are chosen (independent of whether these probabilities are [97%, 95%, 91%] or [80%, 40%, 30%]). In one example, multiple different types of breast cancer exist. If the patient suffers from a “new type” of breast cancer, it might be that the calculated probability related to “breast cancer” is not very high (not around 95%), however, it will be one of the highest. The level of probability required can thus be set according to the individual need.

Accordingly, in a particular embodiment the presently described methods determine a set of probabilities for a patient having one or more diseases after which one of skill, such as a clinician or diagnostician selects from among those probabilities. In a particular embodiment, this level of probability required for this selection step is according to a pre-determined cut off.

Following the determination that a patient has a high probability of having a particular disease, and before a treatment can be applied, it might be necessary, in particular embodiments to confirm the actual disease, e.g via an imaging method such as a computed tomography scan (CT scan). The present method allows for performing targeted imaging without needing to do a full body scan, though in particular embodiments, a fully body scan is required.

If analysis of a patient sample with the method described above results in an output with a high probability of certain disease localization, standard treatments of this identified disease can be provided. In particular embodiments, a further diagnostic step with imaging methods can be or precede or be the treatment step to classify the stage of malignancy and to decide which treatment is appropriate, or in certain embodiments if treatment is necessary at the particular time.

In particular embodiments, a further diagnostic step can include a mammography and/or ultrasound in case of breast cancer to determine the appropriate therapy which is then provided including but not limited to surgery, chemotherapy, radiation, antibody therapy and/or antihormone therapy.

In other particular embodiments, a further diagnostic step can include a colonoscopy and/or ultrasound and/or X-ray in case of colorectal cancer to determine the appropriate therapy which is then provided, including but not limited to surgery, chemotherapy, radiation and/or antibody therapy.

In some embodiments, a further diagnostic step can include ultrasound and/or white light cystoscopy in case of urinary bladder cancer to determine the appropriate therapy which is provided including but not limited to surgery, chemotherapy, radiation and/or antibody therapy.

In other particular embodiments, a further diagnostic step can include X-ray and/or computer tomography and/or bronchoscopy in case of lung cancer to determine the appropriate therapy which is provided including but not limited to surgery, chemotherapy, radiation and/or antibody therapy.

In other particular embodiments, a further diagnostic step can include different kinds of endoscopy of stomach and oesophagus in case of stomach cancer to determine the appropriate therapy which is provided including but not limited to surgery, chemotherapy, radiation and/or antibody therapy.

In still other particular embodiments, a further diagnostic step can include computer tomography, magnetic resonance imaging, and ultrasound in case of kidney, ovarian, or cervical cancer (among many other cancers that can be imaged accordingly) to determine the appropriate therapy which is provided including but not limited to surgery, chemotherapy, radiation, antihormone, and/or antibody therapy. In the case of endometrium cancer, hysteroscopy can be used.

In particular embodiments, the described methods determine that the patient has a high probability of having leukaemia. In such embodiments, a further diagnostic step might include further lab parameters (such as blood test and accompanying cytology to define the appropriate therapy that is provided such as but not limited to chemotherapy, antibody therapy and/or allogeneic stem cell transplantation.

In other embodiments, a further diagnostic step can include histological and immune histological examination of tissue in case of lymphoma to define the appropriate therapy that is provided such as but not limited to radiation, chemotherapy, antibody therapy and/or autologous stem cell transplantation.

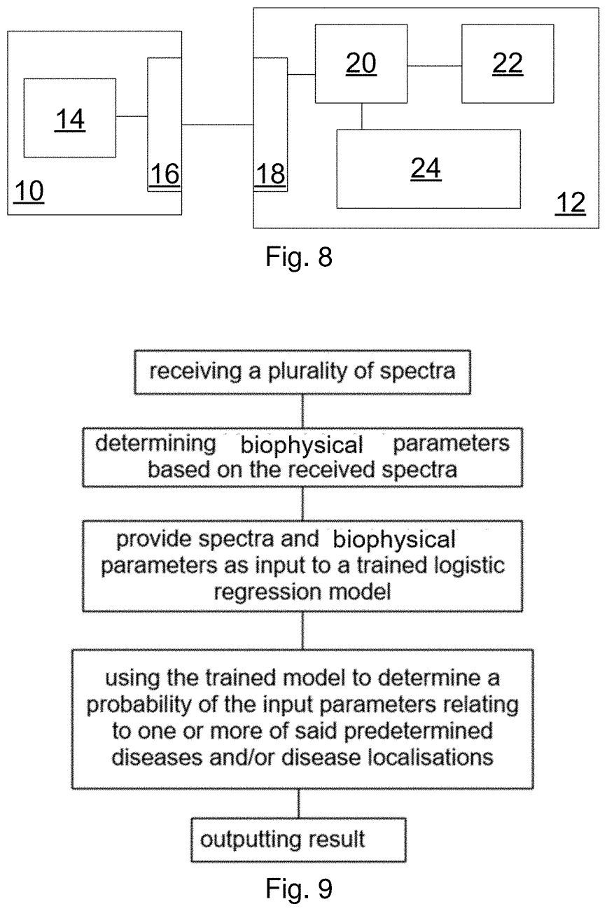

FIG. 8 depicts a system comprising an ESR spectrometer 10 and a computing device 12. The ESR spectrometer comprises an experimental section in which spectra are acquired in the manner discussed above from samples entered into a sample chamber. Acquired spectra are output to the computing device 12 via the output interface 16 of the ESR spectrometer and the input interface 18 of the computing device 12. The computing device 12 comprises a processor 20 and a memory 22. The memory 22 stores program instructions for execution by the processor 20. The program instruction cause the processor 20 to perform the methods described herein when executed by the processor 20. The computing device 12 further comprise an output device 24. The output device 24 may be a display for displaying a determination result to the user or may be an electronic output interface that allows results to be transmitted to other devices. Any such transmission may take place using wireless or wired means.

FIG. 9 is a flowchart of an embodiment of the invention. In a first step a plurality of spectra acquired from a corresponding plurality of aliquots that contain a biophysiological carrier protein is received at a computing device. At least one of a concentration of a spin probe and a concentration of a polar reagent varies between the aliquots. Subsequently biophysical parameters are determined based on the received spectra. At least parts of the received spectra and the biophysical parameters are applied to a trained logistic regression model as inputs. The logistic regression model is trained to determine a probability of the applied input parameters relating to one or more of a plurality of predetermined diseases and/or disease localisations. The trained model is used to determine a probability of the input parameters relating to one or more of said predetermined diseases and/or disease localisations. Results of the determination of a probability are output.

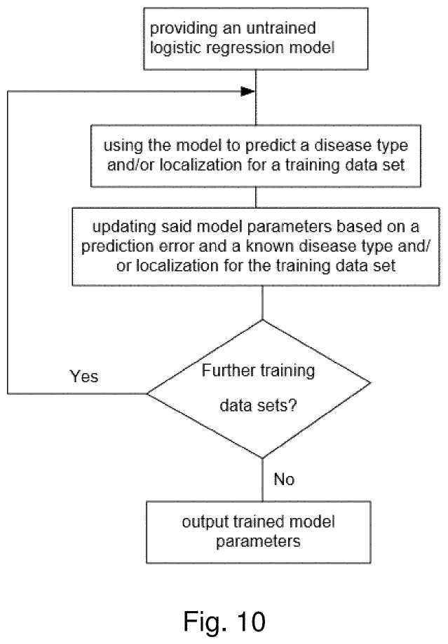

FIG. 10 is a flowchart of a method of training a logistic regression model for determining a probability of applied input parameters relating to one or more of a plurality of predetermined diseases and/or disease localizations. An untrained logistic regression model comprising model parameters is provided to a computing device in a first step. The model is used to predict a disease type and/or localization for a training data set for which disease type and/or localization are already known from clinical diagnosis. The model parameters are then updated based on a prediction error and the known disease type and/or localization for said training data set. If further training data sets are available they are sequentially used for making a predication as discussed above and for correcting the model parameters based on a calculated predication error. If no further training data sets are available trained model parameters are output. Whilst certain embodiments have been described, these embodiments have been presented by way of example only, and are not intended to limit the scope of the inventions. Indeed, the novel devices, and methods described herein may be embodied in a variety of other forms; furthermore, various omissions, substitutions and changes in the form of the devices, methods and products described herein may be made without departing from the spirit of the inventions. The accompanying claims and their equivalents are intended to cover such forms or modifications as would fall within the scope and spirit of the inventions.

We claim:

1. A method for treatment of a malignancy in a patient, comprising:
providing a sample from a patient comprising serum albumin;
combining, in aliquots of the sample, a spin probe and a polar reagent comprising alcohol or dimethyl sulfoxide (DMSO), wherein at least one of the concentration of the spin probe and the concentration of the polar reagent varies between the aliquots;
detecting a plurality of electron spin resonance spectroscopy (ESR) spectra from the aliquots;
determining from the plurality of ESR spectra, a plurality of biophysical parameters selected from the group consisting of: a spectral component from spin probe bound to albumin with a high binding affinity; a spectral component from spin probe bound to albumin with a low binding affinity; a spectral component from free spin probe molecules; a spectral components from free spin probe in micelles; and a spectral component from spin probe on lipid-fraction of serum;
applying the determined biophysical parameters, or parts thereof, as an input to a trained logistic regression model, the logistic regression model trained to determine a probability of applied input parameters relating to one or more of a plurality of predetermined malignancies and locations thereof;
determining the presence of the malignancy and location thereof from the determined probability; and
providing to the patient a treatment appropriate to the determined malignancy.

2. The method of claim 1, wherein the biophysical parameters are selected from the group consisting of: polarity surrounding a spin label in one or more high affinity spectral components; spin probe ordering; spin probe effective correlation time; spectral intensity; and a spectral geometry factor.

3. The method of claim 1, wherein the malignancy is a colorectal cancer, enterogastrologic cancer, gynecologic cancer, kidney cancer, leukemia, lung cancer, lymphoma, breast cancer, pancreatic cancer, prostate cancer, or stomach cancer.

4. The method of claim 1, wherein the treatment is or is preceded by diagnostic evaluation, surgery, chemotherapy, radiation, antibody therapy and/or antihormone therapy.

5. The method of claim 4, wherein the diagnostic evaluation is ultrasound imaging, radiological imaging, biopsy evaluation by microscopy, bronchospopy, colonoscopy, endoscopy, hysteroscopy, and histology.

6. The method of claim 5, wherein the radiological imaging is X-ray, computed tomography, magnetic resonance imaging, and mammography.

7. The method of claim 1, wherein the output of the probability of disease is equal to or greater than a cut-off.

8. The method of claim 7, wherein the malignancy is a lymphoma, and wherein the cut-off is a sensitivity between about 82% to about 92% and/or a specificity between about 88% to about 98%.

9. The method of claim 7, wherein the malignancy is a pancreatic cancer, and wherein the cut-off is a sensitivity between about 69% to about 79% and/or a specificity between about 89% to about 99%.

10. The method of claim 7, wherein the malignancy is a colo-rectal cancer, and wherein the cut-off is a sensitivity between about 54% to about 64% and/or a specificity between about 85% to about 95%.

11. The method of claim 7, wherein the malignancy is a leukemia, and wherein the cut-off is a sensitivity between about 57% to about 67% and/or a specificity between about 86% to about 96%.

12. The method of claim 7, wherein the malignancy is a prostate cancer, and wherein the cut-off is a sensitivity between about 65% to about 75% and/or a specificity between about 81% to about 91%.

13. The method of claim 7, wherein the malignancy is a breast cancer, and wherein the cut-off is a sensitivity between about 73% to about 83% and/or a specificity between about 85% to about 95%.

14. The method of claim 1, wherein the sample is a blood sample from the patient.
